SEQUENCE LISTING (1 sequence)

SEQ ID NO: 1 - protein, 19 aa, Homo Sapiens
DLDLEMLAPYIPMDDDFQL

(19) United States
(12) Patent Application Publication
(10) Pub. No.: US 2017/0240511 A1
(43) Pub. Date: Aug. 24, 2017

(54) SUBSTITUTED PYRIDINE INHIBITORS OF HIF PROLYL HYDROXYLASE

(71) Applicants: Fez UJJAINWALLA, Kenilworth, NJ (US); John Qiang TAN, Princeton, NJ (US); Qun DANG, Kenilworth, NJ (US); Christopher J. SINZ, Rahway,, NJ (US); Ming WANG, Rahway, NJ (US); Jiaqiang CAI, Shanghai (CN); Xiaoxing DU, Shanghai (CN); Yili CHEN, Rahway, NJ (US); MERCK SHARP & DOHME CORP., RAHWAY, NJ (US)

(72) Inventors: FEZ UJJAINWALLA, HOBOKEN, NJ (US); JOHN QIANG TAN, PRINCETON, NJ (US); QUN DANG, WESTFIELD, NJ (US); CHRISTOPHER J. SINZ, MIDDLETOWN, NJ (US); MING WANG, BELLE MEAD, NJ (US); JIAQIANG CAI, SHANGHAI (CN); XIAOXING DU, SHANGHAI (CN); YILI CHEN, HILLSBOROUGH, NJ (US)

(73) Assignee: MERCK SHARP & DOHME CORP., RAHWAY, NJ (US)

(21) Appl. No.: 15/517,553

(22) PCT Filed: Oct. 8, 2015
(86) PCT No.: PCT/US15/54605

§ 371 Date: Apr. 7, 2017

(30) Foreign Application Priority Data
Oct. 10, 2014 (CN) PCT/CN2014/088320

(51) Int. Cl. C07D 213/82; C07D 401/04

(52) U.S. Cl. CPC C07D 213/82; C07D 401/04

(57) ABSTRACT

The present invention concerns compounds of formula I that inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### BACKGROUND OF THE INVENTION

The insufficient delivery of oxygen to cells and tissues is associated with anemia, which is defined as a deficiency in the blood's oxygen-carrying capacity, and ischemia, in which restrictions in blood supply are caused by a constriction or blockage of blood vessels. Anemia can be caused by the loss of red blood cells (hemorrhage), excessive red blood cell destruction (hemolysis) or deficiencies in erythropoiesis (production of red blood cells from precursors found in the bone marrow). The symptoms of anemia can include weakness, dizziness, fatigue, pallor, impairment of cognitive function and a general reduction in quality of life. Chronic and/or severe anemia can lead to the exacerbation of myocardial, cerebral or peripheral ischemia and to heart failure. Ischemia is defined as an absolute or relative shortage of oxygen to a tissue or organ and can result from disorders such as atherosclerosis, diabetes, thromboembolisms, hypotension, etc. The heart, brain and kidney are especially sensitive to ischemic stress caused by low blood supply.

The primary pharmacological treatment for anemia is administration of some variant of recombinant human erythropoietin (EPO). For anemias associated with kidney disease, chemotherapy-induced anemia, anemia from HIV-therapy or anemia due to blood loss, recombinant EPO is administered to enhance the supply of the hormone, correct the shortage of red blood cells and increase the blood's oxygen-carrying capacity. EPO replacement is not always sufficient to stimulate optimal erythropoiesis (e.g., in patients with iron processing deficiencies) and has associated risks.

Hypoxia-inducible factor (HIF) has been identified as a primary regulator of the cellular response to low oxygen. HIF is a heterodimeric gene transcription factor consisting of a highly regulated α-subunit (HIF-α) and a constitutively expressed β-subunit (HIF-β, also known as ARNT, or aryl hydrocarbon receptor nuclear transporter). HIF target genes are reported to be associated with various aspects of erythropoiesis (e.g., erythropoietin (EPO) and EPO receptor), glycolysis and angiogenesis (e.g., vascular endothelial growth factor (VEGF)). Genes for proteins involved in iron absorption, transport and utilization as well as heme synthesis are also targets of HIF.

Under normal oxygenation, HIF-α is a substrate in a reaction with molecular oxygen, which is catalyzed by a family of iron(II)-, 2-ketoglutarate- and ascorbate-dependent dioxygenase enzymes called PHD-1 (EGLN2, or egg laying abnormal 9 homolog 2, PHD2 (EGLN1), and PHD3 (EGLN3). Proline residues of HIF-α are hydroxylated (e.g., Pro-402 and Pro-564 of HIF-1α) and the resulting product is a target of the tumor suppressor protein von-Hippel Lindau, a component of an E3 ubiquitin ligase multiprotein complex involved in protein ubiquitination. Under low oxygenation, the HIF-α hydroxylation reaction is less efficient and HIF-α is available to dimerize with HIF-β. HIF dimers are translocated to the cell nucleus here they bind to a hypoxia-responsive enhancer element of HIF target genes.

Cellular levels of HIF are known to increase under conditions of hypoxia and after exposure to hypoxia mimetic agents. The latter includes, but is not limited to, specific metal ions (e.g., cobalt, nickel, manganese), iron chelators (e.g., desferrioxamine) and analogs of 2-ketoglurate (e.g., N-oxalyl glycine). The compounds of the present invention inhibit the HIF prolyl hydroxylases (PHD-1, PHD-2, PHD-3) and can also serve to modulate HIF levels. These compounds therefore have utility for the treatment and/or prevention of disorders or conditions where HIF modulation is desirable, such as anemia and ischemia. As an alternative to recombinant erythropoietin therapy, the compounds of the present invention provide a simpler and broader method for the management of anemia.

### SUMMARY OF THE INVENTION

The present invention concerns compounds of formula I

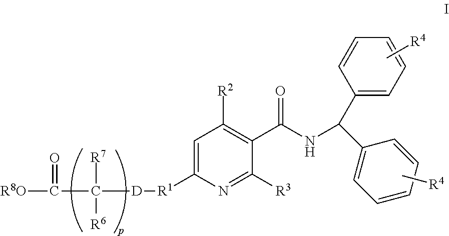

which inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### DETAILED DESCRIPTION OF THE INVENTION

The present invention provides compounds of formula I or stereoisomers or pharmaceutically acceptable salts thereof:

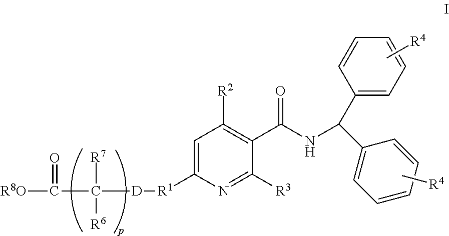

R¹ is —CONR⁵—, C₃₋₁₂cycloalkyldiyl, or a heteroaryldiyl selected from isoxazolyldiyl, tetrazolyldiyl, pyrazolyldiyl, imidazolyldiyl, oxazolyldiyl, thiazolyldiyl, pyridinyldiyl, pyradizinyldiyl, and pyrimidinyldiyl;
R⁵ is hydrogen, C₁₋₃alkyl, or C₁₋₃alkoxy;
R⁸ is hydrogen or C₁₋₃alkyl;
p is 0, 1, 2 or 3;
R² and R³ are each independently selected from hydrogen, hydroxy, —OR, —OCOR, —OCOOR, —OCONHR, and C₁₋₆alkyl;
R is independently selected from hydrogen, C₁₋₁₀ alkyl, —C₁₋₅ alkylaryl, —CR′R′—OCO—C₁₋₁₀ alkyl, and —CR′R′—OCO—O—C₁₋₁₀ alkyl;
R′ and R″ are independently selected from hydrogen and C₁₋₁₀ alkyl;
D is selected from a bond, C₃₋₁₂cycloalkyldiyl, C₃₋₁₂cycloheteroalkyldiyl, aryldiyl, and heteroaryldiyl;
R⁴, R⁶, and R⁷ are each independently selected from

hydrogen,

halogen,

C₁₋₁₀ alkyl,

C₂₋₁₀ alkenyl,

C₂₋₁₀ alkynyl,

C₁₋₁₀ alkylamino,

arylC₀₋₁₀ alkyl,

C₃₋₈ cycloalkyl C₀₋₁₀ alkyl,

C₃₋₈ heteroaryl C₀₋₁₀ alkyl,

C₃₋₈ heterocycloalkyl C₀₋₁₀ alkyl,

C₁₋₁₀ alkoxy, and

hydroxyC₀₋₁₀alkyl, wherein R⁶ and R⁷ may optionally join together with the carbon to which they are attached to form a 3 to 8 membered ring;
wherein R⁴, R⁶, R⁷, and D are each optionally substituted with 0, 1, or 2 R⁹ substituents selected from:

hydrogen,

halogen,

(carbonyl)₀₋₁C₁₋₁₀ alkyl,

(carbonyl)₀₋₁C₂₋₁₀ alkenyl,

(carbonyl)₀₋₁C₂₋₁₀ alkynyl,

amino C₁₋₁₀alkyl,

C₁₋₁₀ alkylamino C₀₋₁₀ alkyl,

cyano,

nitro,

C₁₋₆haloalkyl,

perfluoroC₁₋₆alkyl, and

perfluoroC₁₋₆alkoxy.

Another embodiment of the invention provides compounds of Formula II or stereoisomers thereof, or pharmaceutically acceptable salts thereof:

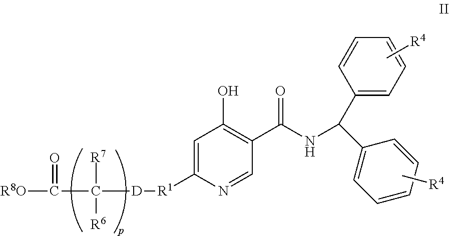

R¹ is —CONR⁵— or a heteroaryldiyl selected from isoxazolyldiyl, tetrazolyldiyl, pyrazolyldiyl, imidazolyldiyl, oxazolyldiyl, thiazolyldiyl, pyridinyldiyl, pyradizinyldiyl, and pyrimidinyldiyl;
R⁵ is hydrogen, C₁₋₃alkyl, or C₁₋₃alkoxy;
R⁸ is hydrogen or C₁₋₃alkyl;
p is 0, 1, 2 or 3;
D is selected from a bond and C₃₋₁₂cycloalkyldiyl, and
R⁴, R⁶, and R⁷ are each independently selected from

hydrogen,

halogen,

C₁₋₁₀ alkyl, and

hydroxyC₀₋₁₀alkyl, wherein R⁶ and R⁷ may optionally join together with the carbon to which they are attached to form a 3 to 8 membered ring.

Another embodiment of the invention, is a compound of Formulas I and II, or stereoisomers thereof, or pharmaceutically acceptable salts thereof wherein:
R¹ is —CONH— or a heteroaryldiyl selected from tetrazolyldiyl, and pyrazolyldiyl;
R⁵ is hydrogen;
R⁸ is hydrogen or C₁₋₃alkyl;
p is 0, 1, 2 or 3;
D is selected from a bond, cyclopropyl, cyclobutyl, and bicyclo[1.1.1]pentyl; and
R⁴, R⁶, and R⁷ are each independently selected from

hydrogen,

halogen,

C₁₋₃ alkyl, and

hydroxy, wherein R⁶ and R⁷ may optionally join together with the carbon to which they are attached to form a 3 to 8 membered ring.

Another embodiment, is a compound of Formulas I and II, or stereoisomers thereof, or pharmaceutically acceptable salts thereof wherein: R⁸ is hydrogen, methyl, or ethyl;
R⁴ is hydrogen or chloro; R⁶ and R⁷ are each independently selected from hydrogen, methyl, and hydroxyl, wherein R⁶ and R⁷ may optionally join together with the carbon to which they are attached to form a 3 membered ring.

Representative compounds of the instant invention include, but are not limited to, the following compounds and their pharmaceutically acceptable salts and their stereoisomers thereof:
1-(5-(Benzhydrylcarbamoyl)-4-hydroxypyridin-2-yl)-1H-pyrazole-4-carboxylic acid;
methyl 3-(5-(benzhydrylcarbamoyl)-4-methoxypicolinamido)propanoate;
methyl 3-(5-(benzhydrylcarbamoyl)-4-hydroxypicolinamido)-2-methylpropanoate;
ethyl 1-((5-(benzhydrylcarbamoyl)-4-hydroxypicolinamido)methyl)cyclopropanecarboxylate;
ethyl 3-(5-(benzhydrylcarbamoyl)-4-hydroxypicolinamido)-2,2-dimethylpropanoate;
(1S,2S)-ethyl 2-(5-(benzhydrylcarbamoyl)-4-hydroxypicolinamido)cyclopropanecarboxylate;
(1R,2R)-ethyl 2-(5-(benzhydrylcarbamoyl)-4-hydroxypicolinamido)cyclopropanecarboxylate;
trans-3-(5-(benzhydrylcarbamoyl)-4-hydroxypicolinamido)cyclobutanecarboxylic acid;
3-(5-(benzhydrylcarbamoyl)-4-hydroxypicolinamido)propanoic acid;
3-(5-(benzhydrylcarbamoyl)-4-hydroxypicolinamido)-2-methylpropanoic acid;
1-((5-(benzhydrylcarbamoyl)-4-hydroxypicolinamido)methyl)cyclopropanecarboxylic acid;
4-(5-(benzhydrylcarbamoyl)-4-hydroxypicolinamido)butanoic acid;
3-(5-(benzhydrylcarbamoyl)-4-hydroxypicolinamido)-2-hydroxypropanoic acid;
3-(5-(benzhydrylcarbamoyl)-4-hydroxypicolinamido)-2,2-dimethylpropanoic acid;
(1S,2S)-2-(5-(benzhydrylcarbamoyl)-4-hydroxypicolinamido)cyclopropanecarboxylic acid;
(1R,2R)-2-(5-(benzhydrylcarbamoyl)-4-hydroxypicolinamido)cyclopropanecarboxylic acid;
3-(5-(benzhydrylcarbamoyl)-4-hydroxypicolinamido)bicyclo[1.1.1]pentane-1-carboxylic acid;
2-(5-(5-(benzhydrylcarbamoyl)-4-hydroxypyridin-2-yl)-1H-tetrazol-1-yl)acetic acid; and
ethyl 1-((5-((bis(4-chlorophenyl)methyl)carbamoyl)-4-hydroxypicolinamido)methyl) cyclopropanecarboxylate.

As used herein except where noted, “alkyl” is intended to include both branched- and straight-chain saturated aliphatic hydrocarbon groups, including all isomers, having the specified number of carbon atoms. Commonly used abbreviations for alkyl groups are used throughout the specification, e.g. methyl may be represented by “Me” or CH₃, ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₆ alkyl” (or “C₁-C₆ alkyl”) for example, means linear or branched chain alkyl groups, including all isomers, having the specified number of carbon atoms. C₁₋₆ alkyl includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl.

The term “halogen” (or “halo”) refers to fluorine, chlorine, bromine and iodine (alternatively referred to as fluoro (F), chloro (Cl), bromo (Br), and iodo (I)).

The term “aryl” refers to aromatic mono- and poly-carbocyclic ring systems, wherein the individual carbocyclic rings in the polyring systems are fused or attached to each other via a single bond. Suitable aryl groups include phenyl, naphthyl, and biphenylenyl.

The term “carbocycle” (and variations thereof such as “carbocyclic” or “carbocyclyl”) as used herein, unless otherwise indicated, refers to (i) a C₃ to C₈ monocyclic, saturated or unsaturated ring or (ii) a C₇ to C₁₂ bicyclic saturated or unsaturated ring system. Each ring in (ii) is either independent of, or fused to, the other ring, and each ring is saturated or unsaturated. The carbocycle may be attached to the rest of the molecule at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A fused bicyclic carbocycle in which one ring is saturated and the other is saturated is a saturated bicyclic ring system. A fused bicyclic carbocycle in which one ring is benzene and the other is saturated is an unsaturated bicyclic ring system. A fused bicyclic carbocycle in which one ring is benzene and the other is unsaturated is an unsaturated ring system. Saturated carbocyclic rings are also referred to as cycloalkyl rings, e.g., cyclopropyl, cyclobutyl, etc. Unless otherwise noted, carbocycle is unsubstituted or substituted with C₁₋₆ alkyl, C₁₋₆ alkenyl, C₁₋₆ alkynyl, aryl, halogen, NH₂ or OH. A subset of the fused bicyclic unsaturated carbocycles are those bicyclic carbocycles in which one ring is a benzene ring and the other ring is saturated or unsaturated, with attachment via any carbon atom that results in a stable compound. Representative examples of this subset include the following:

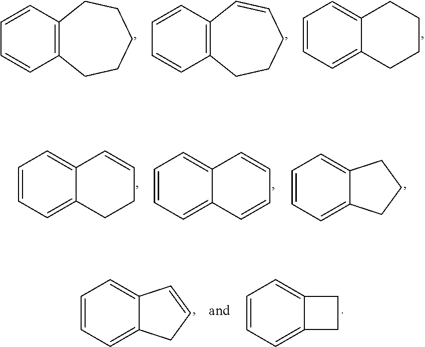

The term “heterocycle” (and variations thereof such as “heterocyclic” or “heterocyclyl”) broadly refers to (i) a stable 4- to 8-membered, saturated or unsaturated monocyclic ring, or (ii) a stable 7- to 12-membered bicyclic ring system, wherein each ring in (ii) is independent of, or fused to, the other ring or rings and each ring is saturated or unsaturated, and the monocyclic ring or bicyclic ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

Non limiting examples of heterocyclylic moieties include, but are not limited to, the following: pyrazolyl, azepanyl, azabenzimidazole, benzoimidazolyl, benzofuryl, benzofurazanyl, benzopyrazolyl, benzothiazolyl, benzothienyl, benzotriazolyl, benzothiophenyl, benzoxazolyl, carbazolyl, carbolinyl, chromanyl, cinnolinyl, furanyl, imidazolyl, indolinyl, indolyl, indolazinyl, indazolyl, isobenzofuryl, isochromanyl, isoindolyl, isoquinolyl, isothiazolyl, isoxazolyl, naphthpyridinyl, oxadiazolyl, oxazolyl, oxazolinyl, isooxazolinyl, oxetanyl, pyranyl, pyrazinyl, pyrazolidinyl, pyrazolyl, pyridazinyl, pyridopyridinyl, pyridazinyl, pyridinyl, pyrimidyl, pyrrolyl, quinazolinyl, quinolyl, quinoxalinyl, quinoxalinyl, tetrahydropyranyl, tetrahydroisoquinolinyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuryl, dihydrobenzothiophenyl, dihydrobenzoxazolyl, dihydrofuryl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, tetrahydroquinolinyl, tetrahydroisoquinolinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuryl, tetrahydrothienyl, tetrahydroquinolinyl, 2,3-dihydrobenzofuryl, 2,3-dihydrobenzo-1,4-dioxinyl, imidazo(2,1-b)(1,3)thiazole, and benzo-1,3-dioxolyl.

Heteroaromatics form another subset of the heterocycles; i.e., the term “heteroaromatic” (alternatively “heteroaryl”) generally refers to a heterocycle as defined above in which the entire ring system (whether mono- or poly-cyclic) is an aromatic ring system. The term “heteroaromatic ring” refers a 5- or 6-membered monocyclic aromatic ring or a 7- to 12-membered bicyclic which consists of carbon atoms and one or more heteroatoms selected from N, O and S. In the case of substituted heteroaryl rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

“Hydroxyalkyl” refers to an alkyl group as described above in which one or more (in particular 1 to 3) hydrogen atoms have been replaced by hydroxy groups. Examples include CH₂OH, CH₂CHOH and CHOHCH₃.

“Alkyldiyl,” “alkenyldiyl,” “alkynyldiyl”, “cycloalkyldiyl”, “aryldiyl”, “heteroaryldiyl” and “heterocycloalkyldiyl” refer to a divalent radical obtained by the removal of one hydrogen atom from an alkyl, alkenyl, alkynyl, cycloalkyl, aryl, heteroaryl, and heterocycloalkyl group, respectively, each of which is as defined above.

Unless expressly stated to the contrary, all ranges cited herein are inclusive. For example, a heterocycle described as containing from “1 to 4 heteroatoms” means the heterocycle can contain 1, 2, 3 or 4 heteroatoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed.

When any variable (e.g., R^{b}, etc.) occurs more than one time in any substituent or in Formulas I-II, its definition in each occurrence is independent of its definition at every other occurrence. Also, combinations of substituents and/or variables are permissible only if such combinations result in stable compounds.

In choosing compounds of the present invention, one of ordinary skill in the art will recognize that the various substituents, i.e. R¹, R², R³, etc., are to be chosen in conformity with well-known principles of chemical structure connectivity.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups can be on the same carbon or on different carbons, so long as a stable structure results. The phrase “optionally substituted with one or more substituents” should be taken to be equivalent to the phrase “optionally substituted with at least one substituent” and in such cases one embodiment will have from zero to three substituents.

### Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formulas I and II are shown without a definitive stereochemistry at certain positions. The present invention includes all stereoisomers of Formulas I and II and pharmaceutically acceptable salts and solvates thereof. Unless specifically mentioned otherwise, reference to one isomer applies to any of the possible isomers. Whenever the isomeric composition is unspecified, all possible isomers are included. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I and II may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)— groups (keto forms) may undergo tautomerism to form hydroxy-CH═C(OH)— groups (enol forms). Both keto and enol forms, individually as well as mixtures thereof, are included within the scope of the present invention.

### Salts

Pharmaceutically acceptable salts include both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical stability, flowability, hydro-scopicity and solubility. The term “pharmaceutically acceptable salts” refers to salts prepared from pharmaceutically acceptable non-toxic bases or acids. When the compound of the present invention is acidic, its corresponding salt can be conveniently prepared from inorganic bases or organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from organic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, dicyclohexylamine, lysine, methyl-glucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from inorganic or organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methane-sulfonic, mucic, nitric, pamoic, pantothenic, phosphoric, succinic, sulfuric, tartaric, p-toluene-sulfonic acid and the like. Preferred are citric, hydrobromic, hydrochloric, maleic, phosphoric, sulfuric, and tartaric acids.

### Solvates

The present invention includes within its scope solvates of compounds of Formula I and II. As used herein, the term “solvate” refers to a complex of variable stoichiometry formed by a solute (i.e., a compound of Formula I or II) or a pharmaceutically acceptable salt thereof and a solvent that does not interfere with the biological activity of the solute. Examples of solvents include, but are not limited to water, ethanol, and acetic acid. When the solvent is water, the solvate is known as hydrate; hydrate includes, but is not limited to, hemi-, mono, sesqui-, di- and trihydrates.

### Prodrugs

The present invention includes within its scope the use of prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of Formula I or II, or with a compound which may not be a compound of Formula I or II, but which converts to a compound of Formula Ior II in vivo after administration to the patient. Conventional procedures for the selection and preparation of suitable prodrug derivatives are described, for example, in “Design of Prodrugs,” ed. H. Bundgaard, Elsevier, 1985.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I or II. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I or II can be prepared without undue experimentation by conventional techniques well known to those skilled in the art or by processes analogous to those described in the Schemes and Examples herein using appropriate isotopically-enriched reagents and/or intermediates.

### Utilities

Compounds of the present invention are inhibitors of hypoxia-inducible factor (HIF) prolyl hydroxylases, and as such are useful in the treatment and prevention of diseases and conditions in which HIF modulation is desirable, such as anemia and ischemia. Compounds of the invention can be used in a selective and controlled manner to induce hypoxia-inducible factor stabilization and to rapidly and reversibly stimulate erythropoietin production and secretion. Accordingly, another aspect of the present invention provides a method of treating or preventing a disease or condition in a mammal, the treatment or prevention of which is effected or facilitated by HIF prolyl hydroxylase inhibition, which comprises administering an amount of a compound of Formula I or II that is effective for inhibiting HIF prolyl hydroxylase. This aspect of the present invention further includes the use of a compound of Formula I or II in the manufacture of a medicament for the treatment or prevention of a disease or condition modulated by HIF prolyl hydroxylase.

In one embodiment is a method of enhancing endogenous production of erythropoietin in a mammal which comprises administering to said mammal an amount of a compound of Formula I that is effective for enhancing endogenous production of erythropoietin.

Another embodiment is a method of treating anemia in a mammal which comprises administering to said mammal a therapeutically effective amount of a compound of Formulas I or II. “Anemia” includes, but is not limited to, chronic kidney disease anemia, chemotherapy-induced anemia (e.g., anemia resulting from antiviral drug regimens for infectious diseases, such as HIV and hepatitis C virus), anemia of chronic disease, anemia associated with cancer conditions, anemia resulting from radiation treatment for cancer, anemias of chronic immune disorders such as rheumatoid arthritis, inflammatory bowel disease, and lupus, and anemias due to menstruation or of senescence or in other individuals with iron processing deficiencies such as those who are iron-replete but unable to utilize iron properly.

Another embodiment is a method of treating ischemic diseases in a mammal, which comprises administering to said mammal a therapeutically effective amount of a compound of Formulas I or II.

### Combination Therapy

Compounds of Formulas I and II may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formulas I or II. When a compound of Formulas I or II is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formulas I or II.

### Route of Administration/Dosage

The compounds of this invention can be administered for the treatment or prevention of afflictions, diseases and illnesses according to the invention by any means that effects contact of the active ingredient compound with the site of action in the body of a warm-blooded animal. For example, administration can be oral, topical, including transdermal, ocular, buccal, intranasal, inhalation, intravaginal, rectal, intracisternal and parenteral. The term “parenteral” as used herein refers to modes of administration which include subcutaneous, intravenous, intramuscular, intraarticular injection or infusion, intrasternal and intraperitoneal. For the purpose of this disclosure, a warm-blooded animal is a member of the animal kingdom possessed of a homeostatic mechanism and includes mammals and birds.

The compounds can be administered by any conventional means available for use in conjunction with pharmaceuticals, either as individual therapeutic agents or in a combination of therapeutic agents. They can be administered alone, but are generally administered with a pharmaceutical carrier selected on the basis of the chosen route of administration and standard pharmaceutical practice.

The dosage administered will be dependent on the age, health and weight of the recipient, the extent of disease, kind of concurrent treatment, if any, frequency of treatment and the nature of the effect desired. Usually, a daily dosage of active ingredient compound will be from about 0.1-2000 milligrams per day. Ordinarily, from 10 to 500 milligrams per day in one or more applications is effective to obtain desired results. These dosages are the effective amounts for the treatment and prevention of afflictions, diseases and illnesses described above, e.g., anemia.

### Pharmaceutical Composition

Another aspect of the present invention provides pharmaceutical compositions which comprises a compound of Formulas I or II and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formulas I or II, additional active ingredient(s), and pharmaceutically acceptable excipients. The pharmaceutical compositions of the present invention comprise a compound represented by Formulas I or II (or a pharmaceutically acceptable salt or solvate thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The active ingredient can be administered orally in solid dosage forms, such as capsules, tablets, troches, dragees, granules and powders, or in liquid dosage forms, such as elixirs, syrups, emulsions, dispersions, and suspensions. The active ingredient can also be administered parenterally, in sterile liquid dosage forms, such as dispersions, suspensions or solutions. Other dosages forms that can also be used to administer the active ingredient as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene gycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfate, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Suitable pharmaceutical carriers are described in Remington's Pharmaceutical Sciences, A. Osol, a standard reference text in this field.

For administration by inhalation, the compounds of the present invention may be conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of Formulas I or II in suitable propellants, such as fluorocarbons or hydrocarbons.

For ocular administration, an ophthalmic preparation may be formulated with an appropriate weight percent solution or suspension of the compounds of Formulas I or II in an appropriate ophthalmic vehicle, such that the compound is maintained in contact with the ocular surface for a sufficient time period to allow the compound to penetrate the corneal and internal regions of the eye.

Useful pharmaceutical dosage-forms for administration of the compounds of this invention include, but are not limited to, hard and soft gelatin capsules, tablets, parenteral injectables, and oral suspensions.

A large number of unit capsules are prepared by filling standard two-piece hard gelatin capsules each with 100 milligrams of powdered active ingredient, 150 milligrams of lactose, 50 milligrams of cellulose, and 6 milligrams magnesium stearate.

A mixture of active ingredient in a digestible oil such as soybean oil, cottonseed oil or olive oil is prepared and injected by means of a positive displacement pump into gelatin to form soft gelatin capsules containing 100 milligrams of the active ingredient. The capsules are washed and dried.

A large number of tablets are prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

A parenteral composition suitable for administration by injection is prepared by stirring 1.5% by weight of active ingredient in 10% by volume propylene glycol. The solution is made to volume with water for injection and sterilized.

An aqueous suspension is prepared for oral administration so that each 5 milliliters contain 100 milligrams of finely divided active ingredient, 100 milligrams of sodium carboxymethyl cellulose, 5 milligrams of sodium benzoate, 1.0 grams of sorbitol solution, U.S.P., and 0.025 milliliters of vanillin.

The same dosage forms can generally be used when the compounds of this invention are administered stepwise or in conjunction with another therapeutic agent. When drugs are administered in physical combination, the dosage form and administration route should be selected depending on the compatibility of the combined drugs. Thus the term coadministration is understood to include the administration of the two agents concomitantly or sequentially, or alternatively as a fixed dose combination of the two active components.

Compounds of the invention can be administered as the sole active ingredient or in combination with a second active ingredient, including other active ingredients known to be useful for improving the level of erythropoietin in a patient.

### Abbreviations Used in the Description of the Preparation of the Compounds of the Present Invention:

˜Approximately
AcOH Acetic acid
Ag20 Silver oxide
AIBN 2,2′-azobis(2-methylpropionitrile)
Aq Aqueous
Bn Benzyl
BnBr Benzylbromide
BnCl Benzylchloride
BnOH Benzylalcohol
Boc₂O or di-tert-butyl dicarbonate
BOC₂O
Brine Saturated aqueous sodium chloride solution
CDI Carbonyl diimidazole
DBU 1,8-diazabicyclo[5.4.0]undec-7-ene
DCC N,N′-dicyclohexylcarbodiimide
DEAD diethylazodicarboxylate
DCM Dichloromethane
DIPEA N,N-diisopropylethylaime
DMA Dimethylacetamide
DMAP 4-N,N-dimethylaminopyridine
DMF N,N-dimethylformamide
DMSO Dimethyl sulfoxide
DPPA Diphenyl phosphoryl azide
EDC or EDCI 1-(3-dimethylaminopropyl)-3-ethylcarboiimide hydrogenchloride salt
EtOAc or EA Ethyl acetate
Et (et) Ethyl
EtOH Ethanol
Et₂O or ether Diethyl ether
Et₃N triethylamine
g Gram
h or hr Hour
HATU O-(7-Azabenzotriazol-1-yl)-N,N,N′,N′-tetramethyluronium hexafluorophosphate
HCl Hydrochloric acid
HOBt 1-hydroxybenzatriazole
HPLC High-performance liquid chromatography
i-propanol Isopropyl alcohol
i-PrOH or IPA Isopropyl alcohol
K2CO3 Pₒtassium carbonate
KOH Potassium hydroxide
LCMS Liquid chromatography mass spectrometry
LiOH Lithium hydroxide
Mg Milligrams
mL Milliliters
mmol Millimole
MeCN Acetonitrile
MeOH Methanol
min Minutes
ms or MS Mass spectrum
μg Microgram(s)
μL Microliters
NaOEt Sodium ethoxide
NaOMe Sodium methoxide
Na₂SO₄ Sodium sulfate
NBS N-bromosuccinimide
NHAc Acetamido
NHCbz Benzyloxycarboxamido
NaOH Sodium hydroxide
NaN₃ Sodium azide
NH₄OH ammonium hydroxide
NMP N-methylpyrrolidone
Pd/C Palladium on carbon
PdCl₂(dppf) [1,1′-Bis(diphenylphosphino)ferrocene]dichloropalladium(II)
Pd(OH)2 Palladium hydroxide
Pd(PPh₃)₄ Palladium tetrakis(triphenylphosphine)
PE Petroleum ether
PhLi Phenyl lithium
PG Protecting group
Ph Phenyl group
PMB Para-methoxybenzyl
PPTS Pyridinium Para-toluenesulfonate
PPh₃ Triphenyphosphine
Rₜ Retention time
RT or rt Room temperature
SOCl₂ Thionyl chloride
′Bu Tert-butyl
TEA Triethylamine
TFA Trifluoroacetic acid
THF Tetrahydrofuran
TBTU O-(Benzotriazol-1-yl)-N,N,N′,N′-tetramethyluronium tetrafluoroborate
TMS Trimethylsilyl
TMSC₁ Trimethylsilyl chloride
TsCl Para-toluenesulfonyl chloride

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound in place of multiple substituents which are allowed under the definitions of Formula I or II defined previously.

### General Experimental Comments

Reactions sensitive to moisture or air were performed under nitrogen using anhydrous solvents and reagents. The progress of reactions was determined by either analytical thin layer chromatography (TLC) performed with E. Merck (EMD Millipore, Billerica Mass.) precoated TLC plates, silica gel 60E-254, layer thickness 0.25 mm or liquid chromatography-mass spectrum (LC-MS). Mass analysis was performed on a Waters Micromass® ZQ™ (Waters Corporation, Milford, Mass.) with electrospray ionization in positive ion detection mode. High performance liquid chromatography (HPLC) was conducted on an Agilent 1100 series HPLC on Waters C₁₈ XTerra® 3.5 μm 3.0×50 mm column with gradient 10:90-100 v/v CH₃CN/H₂O+v 0.05% TFA over 3.75 min then hold at 100 CH₃CN+v 0.05% TFA for 1.75 min; flow rate 1.0 mL/min, UV wavelength 254 nm). Concentration of solutions was carried out on a rotary evaporator under reduced pressure. Flash chromatography was performed using a Biotage® Flash Chromatography apparatus (Biotage, Charlotte, N.C.) on silica gel (32-63 mM, 60 Å pore size) in pre-packed cartridges. ¹H-NMR spectra were obtained on a 400 or 500 MHz VARIAN® Spectrometer (Varian, Inc. Palo Alto, Calif.) in CDCl₃ or CD₃OD or other solvents as indicated and chemical shifts are reported as δ using the solvent peak as reference and coupling constants are reported in hertz (Hz).

The general synthetic sequence for compounds of Formula I including Ia, Ib, Ic and Id is outlined in Schemes 1-4. In Scheme 1, the synthesis of compounds of Formula Ia started with chloropyridyl carboxylic acid 1 from which amide 2 was obtained through formation of acid chloride and its reaction with amine. The cyanation of chloride 2 with zinc cyanide and palladium catalyst afforded cyanide 3. Tetrazole formation followed by alkylation gave compound 5. Deprotection with hydrobromic acid completed the synthesis of Formula Ia products.

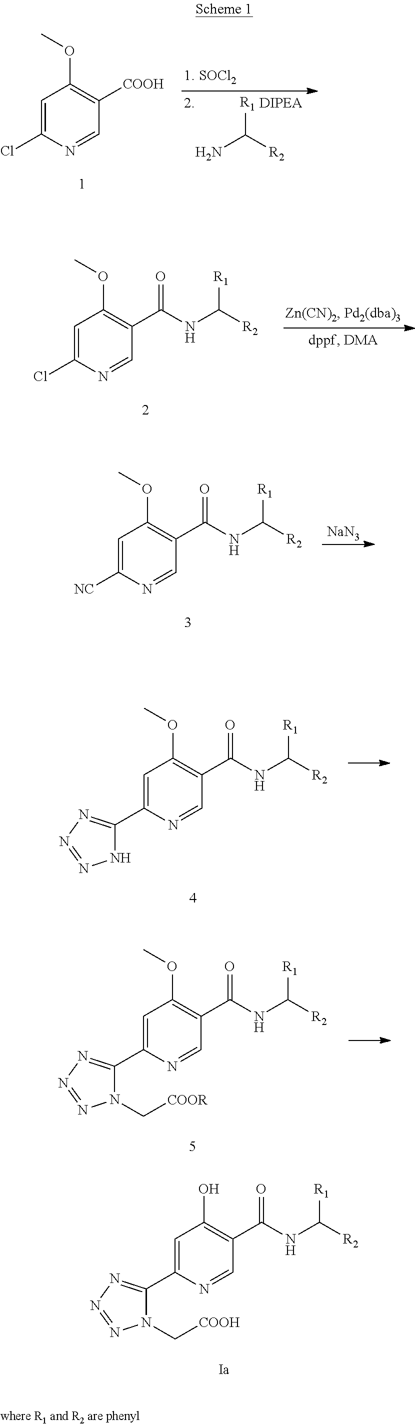

In Scheme 2, Ullmann type reaction of compound 2 with tert-butyl pyrazol-4-carboxylate 5 gave 6, which upon deprotection provided Formula Ib products.

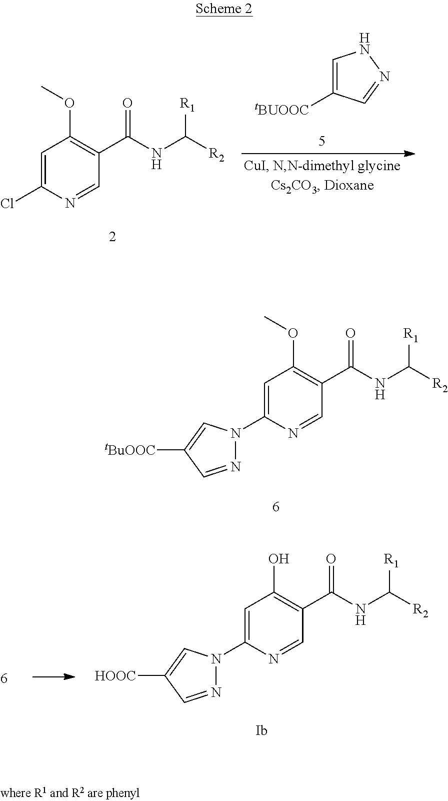

In Scheme 3, carbonylation of compound 2 followed by hydrolysis of methyl ester 7 afforded acid 8. Amide formation between 8 and amino acid 9 gave compound 10. Finally, droprection of 10 provided Formula Ic products.

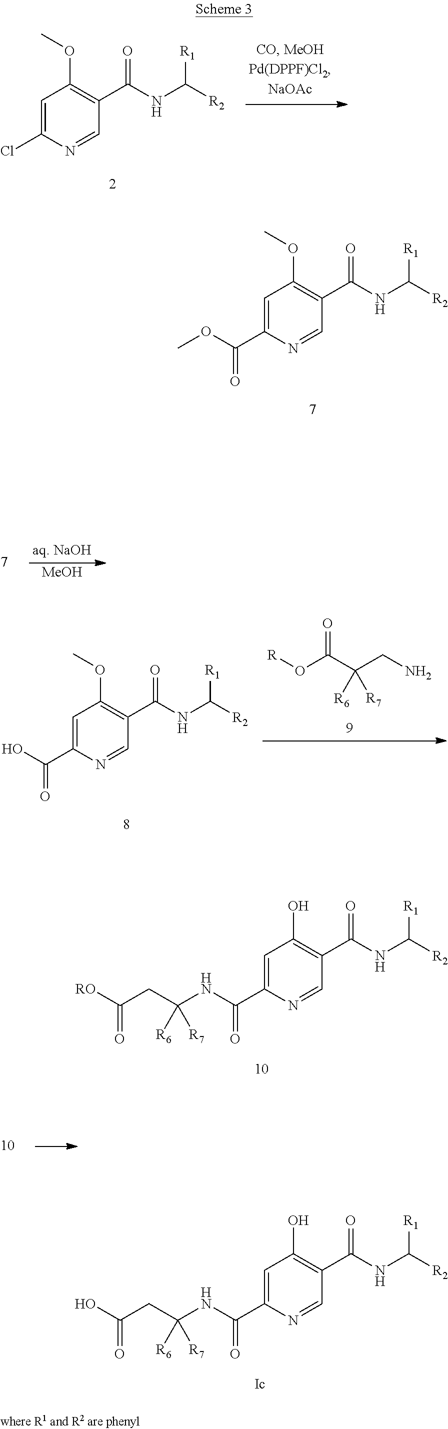

In Scheme 4, in order to modify the amine portion of the right-hand side of the molecule (R₆ and R₇), nicotinic acid 1 was first protected as its tert-butyl ester. After the left-hand side of the molecule was functionalized similarly as those described in Scheme 3, the ^{t}Bu ester was deprotected to give acid 16. Amide formation with amine 17 and hydrolysis of the ethyl ester provided Formula Id products.

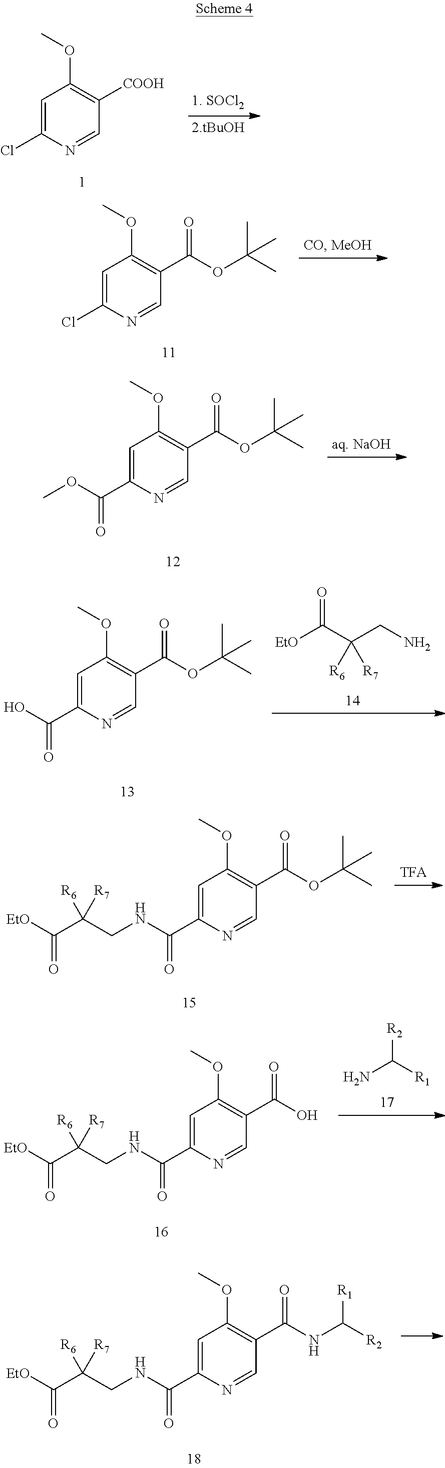

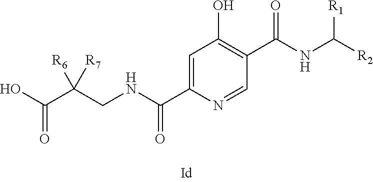

Starting materials useful for the preparation of the compounds in the present invention are known in the art or may be prepared using chemical methodologies known to those skilled in the art.

### EXAMPLE 1

### 1-(5-(Benzhydrylcarbamoyl)-4-hydroxypyridin-2-yl)-1H-pyrazole-4-carboxylic acid

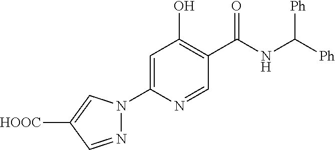 [SMILES: O=C(O)c1cnn(-c2cc(O)c(C(=O)NC(c3ccccc3)c3ccccc3)cn2)c1]

### Step 1: N-Benzhydryl-6-chloro-4-methoxynicotinamide

6-Chloro-4-methoxynicotinic acid (300 mg, 1.5 mmol) was added to thionyl chloride (10 mL) and the mixture was refluxed for 16 h. After concentration, the residue was co-evaporated with dry toluene twice to afford a crude 6-chloro-4-methoxynicotinoyl chloride. After dissolving in DCM (10 ml), the solution was added dropwise to a solution of diphenylmethanamine (270 mg, 1.5 mmol) and DIPEA (750 mg, 5.8 mmol) in DCM (10 ml) at 0° C. After the addition, the reaction mixture was stirred at 0° C. for 2 hours, then quenched by the addition of H₂O (20 mL). The precipitate was collected by suction, and the filter cake was further purified by chromatography on silica gel (eluted by EtOAc /PE=1:1.2) to afford N-benzhydryl-6-chloro-4-methoxynicotinamide. ¹H NMR (DMSO-d₆, 400 MHz) δ 9.11 (d, J=8.6 Hz, 1H), 8.32 (s, 1H), 7.36-7.29 (m, 9H), 7.27-7.21 (m, 2H), 6.28 (d, J=8.6 Hz, 1H), 3.93 (s, 3H). LC/MS (m/z): 353 (M+H)⁺.

### Step 2: tert-Butyl 1-(5-(benzhydrylcarbamoyl)-4-hydroxypyridin-2-yl)-1H-pyrazole-4-carboxylate

To a solution of N-benzhydryl-6-chloro-4-methoxynicotinamide (200 mg, 0.6 mmol) in dioxane (16 mL) were added tert-butyl 1H-pyrazole-4-carboxylate (100 mg, 0.6 mmol), CuI (56 mg, 0.3 mmol), Cs₂CO₃ (550 mg, 1.7 mmol) and N,N-dimethylglycine (64 mg, 0.6 mmol). The mixture was stirred at 120° C. overnight. When TLC showed the reaction completed, the mixture was filtered. The filtrate was diluted with water (20 mL) and extracted with EtOAc (40 mL). The organic layer was washed with brine (20 mL), dried over sodium sulfate and concentrated under vacuum. The residue was purified by prep. TLC (EtOAc/PE=3:1) to afford tert-butyl 1-(5-(benzhydrylcarbamoyl)-4-hydroxypyridin-2-yl)-1H-pyrazole-4-carboxylate. ¹H NMR (CDC₃, 400 MHz) δ 12.87 (s, 1H), 8.88 (s, 1H), 8.50 (s, 1H), 8.02 (s, 1H), 7.55-7.50 (m, 1H), 7.42-7.27 (m, 11H), 6.41 (d, J=7.3 Hz, 1H), 1.57 (s, 9H). LC/MS (m/z): 471 (M+H)⁺.

### Step 3: 1-(5-(Benzhydrylcarbamoyl)-4-hydroxypyridin-2-yl)-1H-pyrazole-4-carboxylic acid

A solution of tert-butyl 1-(5-(benzhydrylcarbamoyl)-4-hydroxypyridin-2-yl)-1H-pyrazole-4-carboxylate (50 mg, 0.1 mmol) in HCl/dioxane (4M, 4 mL) was stirred at room temperature for 3 hours. When TLC showed the reaction completed, the mixture was concentrated under vacuum, and the residue was purified by prep. HPLC (Instrument: Gilson GX281, Column: Gemini 150*25 mm*5 um, Mobile phase A: water with 0.225% HCOOH V/V, Mobile phase B: MeCN, Column temperature: 40° C., Gradient: 45-75% B 10 min, Flow rate: 22 ml/min) to afford 1-(5-(benzhydrylcarbamoyl)-4-hydroxypyridin-2-yl)-1H-pyrazole-4-carboxylic acid. ¹H NMR (DMSO-d₆, 400 MHz) δ 8.98 (brs, 1H), 8.80 (brs, 1H), 8.20 (s, 1H), 7.38-7.36 (m, 9H), 7.29-7.28 (m, 2H), 6.36 (d, J=8.0 Hz, 1H). LC/MS (m/z): 415 (M+H)⁺. Human HIF-PHD2 IC₅₀: 0.875 nM.

### EXAMPLE 2

### Ethyl 3-(5-(benzhydrylcarbamoyl)-4-methoxypicolinamido)propanoate

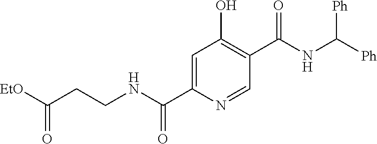 [SMILES: CCOC(=O)CCNC(=O)c1cc(O)c(C(=O)NC(c2ccccc2)c2ccccc2)cn1]

### Step 1: methyl 5-(benzhydrylcarbamoyl)-4-methoxypicolinate

To a 2 L stainless steel autoclave were added N-benzhydryl-6-chloro-4-methoxynicotinamide (4 g, 11.3 mmol), PdCl₂(dppf) (1.66 g, 2.27 mmol), sodium acetate (2.79 g, 34.0 mmol) and MeOH (500 ml). The air in the autoclave was replaced with carbon monoxide, and the pressure was adjusted to 40 atm. Then the reaction mixture was stirred at 120° C. for 16 hours. After cooling, the mixture was filtered, and the filtrate was concentrated under vacuum. The residue was purified by column chromatography on silica gel (eluted with petroleum ether/EtOAc=3:1 to 1:1) to afford methyl 5-(benzhydrylcarbamoyl)-4-methoxypicolinate as white solid. ¹H NMR (CDC₃, 400 MHz) δ 9.32 (s, 1H), 8.20 (d, J=7.5 Hz, 1H), 7.76 (s, 1H), 7.36-7.25 (m, 10H), 6.43 (d, J=7.5 Hz, 1H), 4.06 (s, 3H), 4.00 (s, 3H). LC/MS (m/z): 377 (M+H)⁺.

### Step 2: 5-(benzhydrylcarbamoyl)-4-methoxypicolinic acid

To a solution of methyl 5-(benzhydrylcarbamoyl)-4-methoxypicolinate (300 mg, 0.8 mmol) in MeOH (10 mL) was added aq. NaOH (3 M, 4 mL, 12 mmol). The resulting mixture was stirred at room temperature for 2 hours. When TLC showed the reaction completed, the reaction mixture was concentrated under vacuum, and the residue was diluted with water (5 mL), and acidified with diluted hydrochloric acid to pH=3-4. The precipitate was collected by suction to give 5-(benzhydrylcarbamoyl)-4-methoxypicolinic acid as white solid. ¹H NMR (DMSO-d₆, 400 MHz) δ 9.21 (d, J=8.5 Hz, 1H), 8.59 (br s, 1H), 7.73 (br s, 1H), 7.40-7.34 (m, 8H), 7.30-7.25 (m, 2H), 6.34 (d, J=8.5 Hz, 1H), 3.99 (s, 3H). LC/MS (m/z): 363 (M+H)⁺.

### Step 3: ethyl 3-(5-(benzhydrylcarbamoyl)-4-hydroxypicolinamido)propanoate

To a solution of 5-(benzhydrylcarbamoyl)-4-methoxypicolinic acid (200 mg, 0.56 mmol) in DCM (8 mL) was added ethyl 3-aminopropanoate (172 mg, 1.12 mmol), TBTU (218 mg, 0.68 mmol) and TEA (170 mg, 1.68 mmol). The mixture was stirred at room temperature for 5 hours. When TLC showed the reaction completed, the mixture was diluted with water (15 ml), extracted with DCM (3×20 mL). The organic layers were washed with brine, dried over Na₂SO₄ and concentrated. The crude product was purified by prep. TLC (DCM/MeOH=10:1) to give ethyl 3-(5-(benzhydrylcarbamoyl)-4 methoxypicolinamido)propanoate as a white solid. LC/MS (m/z): 448 (M+H)⁺.

To a solution of ethyl 3-(5-(benzhydrylcarbamoyl)-4-methoxypicolinamido) propanoate (150 mg, 0.23 mmol) and NaI (238 mg,1.65 mmol) in CH₃CN (30 mL) was added TMSC₁ (358 mg, 3.3 mmol). The resulting mixture was stirred at 45° C. for 4 hours. When TLC showed the reaction completed, the reaction mixture was diluted with EtOH (30 mL), the mixture was then concentrated under vacuum. The residue was re-dissolved in EtOAc (60 mL), washed with sat. aqueous Na₂SO₃ and brine, dried over Na₂SO₄ and concentrated to afford ethyl 3-(5-(benzhydrylcarbamoyl)-4-hydroxypicolinamido)propanoate as a white solid. ¹H NMR (DMSO-d₆, 400 MHz) δ 12.44 (br s, 1H), 11.26 (d, J=5.7 Hz, 1H), 9.10-9.04 (m, 1H), 8.36 (s, 1H), 7.34-7.25 (m, 8H), 7.24-7.19 (m, 2H), 7.12 (s, 1H), 6.24 (d, J=7.9 Hz, 1H), 4.03 (q, J=7.1 Hz, 2H), 3.47 (q, J=6.6 Hz, 2H), 2.56 (t, J=6.9 Hz, 2H), 1.14 (t, J=7.1 Hz, 3H). LC/MS (m/z): 448 (M+H)⁺. Human HIF-PHD2 IC₅₀: 92.95 nM.

Examples 3-7 in Table 1 were prepared following the similar procedures described in Example 2 and using the appropriate starting materials.

TABLE 1

| | | | MS m/z (M + 1)⁺ |
|---|---|---|---|
| | | | and human HIF- |
| Example # | Name | Structure | PHD2 IC₅₀ |
| Example 3 | methyl 3-(5- (benzhydrylcarbamoyl)-4- hydroxypicolinamido)-2- methylpropanoate | 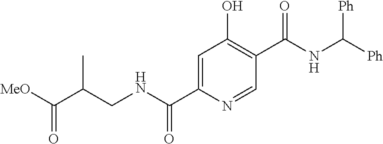 [SMILES: COC(=O)C(C)CNC(=O)c1cc(O)c(C(=O)NC(c2ccccc2)c2ccccc2)cn1] | (M + 1)⁺ 448 IC₅₀ 136.3 nM |
| Example 4 | ethyl 1-((5- (benzhydrylcarbamoyl)-4- hydroxypicolinamido)methyl) cyclopropanecarboxylate | 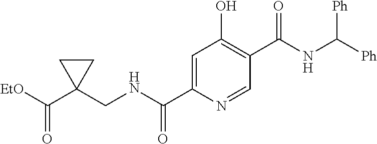 [SMILES: CCOC(=O)C1(CNC(=O)c2cc(O)c(C(=O)NC(c3ccccc3)c3ccccc3)cn2)CC1] | (M + 1)⁺ 474 IC₅₀ 157.9 nM |
| Example 5 | ethyl 3-(5- (benzhydrylcarbamoyl)-4- hydroxypicolinamido)- 2,2-dimethylpropanoate | 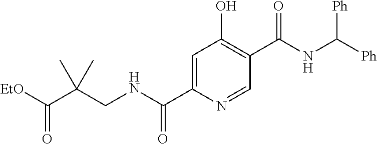 [SMILES: CCOC(=O)C(C)(C)CNC(=O)c1cc(O)c(C(=O)NC(c2ccccc2)c2ccccc2)cn1] | (M + 1)+ 476 IC₅₀ 275.6 nM |
| Example 6 | (1S,2S)-ethyl 2-(5- (benzhydrylcarbamoyl)-4- hydroxypicolinamido) cyclopropanecarboxylate | 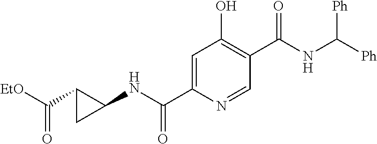 [SMILES: CCOC(=O)[C@H]1C[C@@H]1NC(=O)c1cc(O)c(C(=O)NC(c2ccccc2)c2ccccc2)cn1] | (M + 1)+ 460 IC₅₀ 89.14 nM |
| Example 7 | (1R,2R)-ethyl 2-(5- (benzhydrylcarbamoyl)-4- hydroxypicolinamido) cyclopropanecarboxylate | 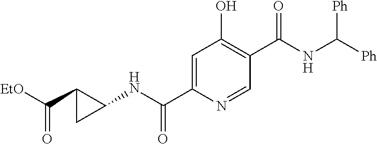 [SMILES: CCOC(=O)[C@@H]1C[C@H]1NC(=O)c1cc(O)c(C(=O)NC(c2ccccc2)c2ccccc2)cn1] | (M + 1)+ 460 IC₅₀ 37.32 nM |

### EXAMPLE 3

### trans-3-(5-(benzhydrylcarbamoyl)-4-hydroxypicolinamido)cyclobutanecarboxylic acid

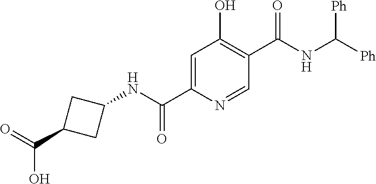 [SMILES: O=C(N[C@H]1C[C@H](C(=O)O)C1)c1cc(O)c(C(=O)NC(c2ccccc2)c2ccccc2)cn1]

### Step 1: trans-methyl 3-(5-(benzhydrylcarbamoyl)-4-methoxypicolinamido) cyclobutanecarboxylate

To a solution of 5-(benzhydrylcarbamoyl)-4-methoxypicolinic acid (100 mg, 0.286 mmol) in DCM (3 mL) were added trans-methyl 3-aminocyclobutanecarboxylate (93 mg, 0.56 mmol), TBTU (109 mg, 0.34 mmol), DIPEA (108 mg, 0.84 mmol). The mixture was stirred at room temperature overnight. When TLC showed the reaction completed, the reaction mixture was diluted with water (15 ml), extracted with DCM (3×10 mL). The organic layer was washed with brine, dried over Na₂SO₄, filtered and concentrated to afford trans-methyl 3-(5-(benzhydrylcarbamoyl)-4-methoxypicolinamido)cyclobutanecarboxylate as a yellow solid.

¹H NMR (CDCl₃, 400 MHz) δ 9.16 (s, 1H), 8.27 (dd, J=7.8, 13.3 Hz, 2H), 7.81 (s, 1H), 7.37-7.24 (m, 11H), 6.44 (d, J=7.7 Hz, 1H), 4.82-4.70 (m, 1H), 4.05 (s, 3H), 3.72 (s, 3H), 3.17-3.09 (m, 1H), 2.79-2.70 (m, 2H), 2.44-2.35 (m, 2H). LC/MS (m/z): 474 (M+H)⁺.

### Step 2: trans-methyl 3-(5-(benzhydrylcarbamoyl)-4-hydroxypicolinamido) cyclobutanecarboxylate

To a solution of trans-methyl 3-(5-(benzhydrylcarbamoyl)-4-methoxypicolinamido) cyclobutanecarboxylate (100 mg, 0.21 mmol) and NaI (158 mg, 1.05 mmol) in CH₃CN (5 mL) was added TMSC₁ (229 mg, 2.1 mmol). The resulting mixture was stirred at 45° C. for 5 hours. When TLC showed the reaction completed, the reaction mixture was diluted with MeOH (20 mL), concentrated under vacuum to remove all the solvents. The residue was re-dissolved in EtOAc (20 mL), washed with sat. aqueous Na₂SO₃ and brine, dried over Na₂SO₄, filtered and concentrated to afford trans-methyl 3-(5-(benzhydrylcarbamoyl)-4-hydroxypicolinamido)cyclobutanecarboxylate as a white solid. ¹H NMR (DMSO-d₆, 400 MHz) δ 12.42 (br s, 1H), 11.16 (br s, 1H), 9.26 (d, J=7.3 Hz, 1H), 8.34 (s, 1H), 7.34-7.26 (m, 8H), 7.25-7.19 (m, 3H), 6.24 (d, J=8.2 Hz, 1H), 4.57-4.45 (m, 1H), 3.61 (s, 3H), 3.11-3.03 (m, 1H), 2.44-2.33 (m, 3H). LC/MS (m/z): 460 (M+H)⁺

### Step 3: trans-3-(5-(benzhydrylcarbamoyl)-4 hydroxypicolinamido)cyclobutanecarboxylic acid

To a solution of trans-methyl 3-(5-(benzhydrylcarbamoyl)-4-hydroxypicolinamido) cyclobutanecarboxylate (63 mg, 0.14 mmol) in MeOH (8 mL) was added aq. NaOH (3 M, 2 mL, 6 mmol). The resulting mixture was stirred at room temperature for 3 hours. When TLC showed the reaction completed, the reaction mixture was concentrated under vacuum, and the residue was diluted with water (5 mL), and acidified with diluted hydrochloric acid to pH=3-4. The precipitate was collected by suction to give trans-3-(5-(benzhydrylcarbamoyl)-4-hydroxypicolinamido)cyclobutanecarboxylic acid as white solid. ¹H NMR (DMSO-d₆, 400 MHz) δ 12.43 (br s, 1H), 11.16 (d, J=7.7 Hz, 1H), 9.27 (d, J=7.3 Hz, 1H), 8.34 (br s, 1H), 7.35-7.27 (m, 9H), 7.26-7.21 (m, 3H), 6.25 (d, J=8.2 Hz, 1H), 4.51 (m, 1H), 3.01-2.92 (m, 1H), 2.43-2.31 (m, 4H). LC/MS (m/z): 446 (M+H)⁺. Human HIF-PHD2 IC₅₀: 1.638 nM.

Examples 9-17 in Table 2 were prepared following the similar procedures described in Example 8 and using the appropriate starting materials.

TABLE 2

| | | | MS m/z (M + 1)⁺ |
|---|---|---|---|
| | | | and human HIF- |
| Examples | Name | Structure | PHD2 IC₅₀ |
| Example 9 | 3-(5- (benzhydrylcarbamoyl)-4- hydroxypicolinamido) propanoic acid | 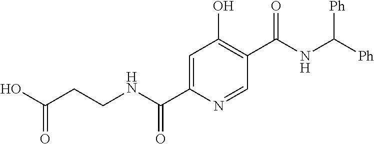 [SMILES: O=C(O)CCNC(=O)c1cc(O)c(C(=O)NC(c2ccccc2)c2ccccc2)cn1] | (M + 1)⁺ 420 IC₅₀ 0.6222 nM |
| Example 10 | 3-(5- (benzhydrylcarbamoyl)-4- hydroxypicolinamido)-2- methylpropanoic acid | 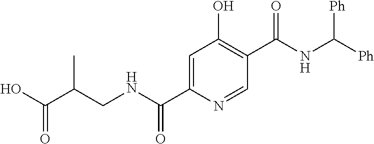 [SMILES: CC(CNC(=O)c1cc(O)c(C(=O)NC(c2ccccc2)c2ccccc2)cn1)C(=O)O] | (M + 1)⁺ 434 IC₅₀ 1.894 nM |
| Example 11 | 1-((5- (benzhydrylcarbamoyl)-4- hydroxypicolinamido)methyl) cyclopropanecarboxylic acid | 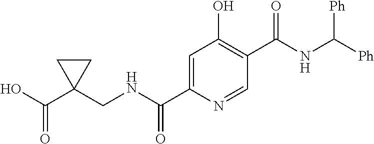 [SMILES: O=C(NCC1(C(=O)O)CC1)c1cc(O)c(C(=O)NC(c2ccccc2)c2ccccc2)cn1] | (M + 1)⁺ 446 IC₅₀ 5.244 nM |
| Example 12 | 4-(5- (benzhydrylcarbamoyl)-4- hydroxypicolinamido) butanoic acid | 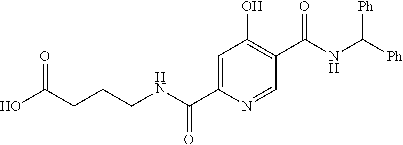 [SMILES: O=C(O)CCCNC(=O)c1cc(O)c(C(=O)NC(c2ccccc2)c2ccccc2)cn1] | (M + 1)⁺ 434 IC₅₀ 1.023 nM |
| Example 13 | 3-(5- (benzhydrylcarbamoyl)-4- hydroxypicolinamido)-2- hydroxypropanoic acid | 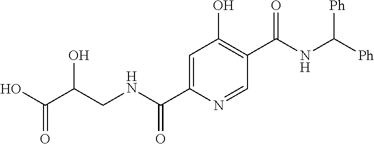 [SMILES: O=C(NCC(O)C(=O)O)c1cc(O)c(C(=O)NC(c2ccccc2)c2ccccc2)cn1] | (M + 1)⁺ 436 IC₅₀ 1.282 nM |
| Example 14 | 3-(5- (benzhydrylcarbamoyl)-4- hydroxypicolinamido)- 2,2-dimethylpropanoic acid | 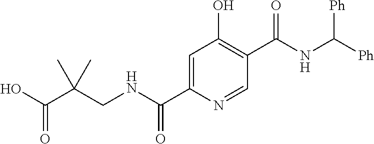 [SMILES: CC(C)(CNC(=O)c1cc(O)c(C(=O)NC(c2ccccc2)c2ccccc2)cn1)C(=O)O] | (M + 1)⁺ 448 IC₅₀ 3.607 nM |
| Example 15 | (1S,2S)-2-(5- (benzhydrylcarbamoyl)-4- hydroxypicolinamido) cyclopropanecarboxylic acid | 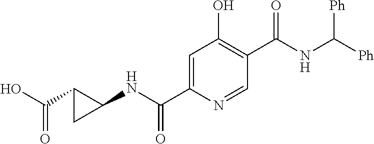 [SMILES: O=C(N[C@H]1C[C@@H]1C(=O)O)c1cc(O)c(C(=O)NC(c2ccccc2)c2ccccc2)cn1] | (M + 1)⁺ 432 IC₅₀ 6.686 nM |
| Example 16 | (1R,2R)-2-(5- (benzhydrylcarbamoyl)-4- hydroxypicolinamido) cyclopropanecarboxylic acid | 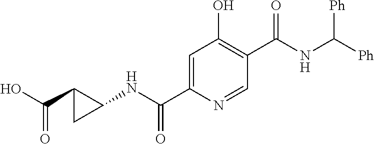 [SMILES: O=C(N[C@@H]1C[C@H]1C(=O)O)c1cc(O)c(C(=O)NC(c2ccccc2)c2ccccc2)cn1] | (M + 1)⁺ 432 IC₅₀ 6.546 nM |
| Example 17 | 3-(5- (benzhydrylcarbamoyl)-4- hydroxypicolinamido)bicyclo [1.1.1]pentane-1- carboxylic acid | 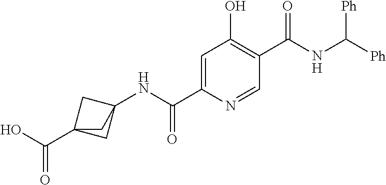 [SMILES: O=C(NC12CC(C(=O)O)(C1)C2)c1cc(O)c(C(=O)NC(c2ccccc2)c2ccccc2)cn1] | (M + 1)⁺ 458 IC₅₀ 23.38 nM |

### EXAMPLE 4

### 2-(5-(5-(benzhydrylcarbamoyl)-4-hydroxypyridin-2-yl)-1H-tetrazol-1-yl)acetic acid

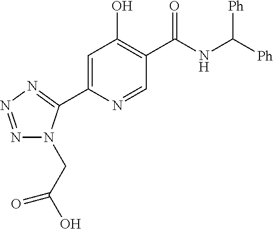 [SMILES: O=C(O)Cn1nnnc1-c1cc(O)c(C(=O)NC(c2ccccc2)c2ccccc2)cn1]

### Step 1: N-benzhydryl-6-cyano-4-methoxynicotinamide

To a solution of N-benzhydryl-6-chloro-4-methoxynicotinamide (50 mg, 0.13 mmol) in DMA (10 mL) were added Zn(CN)₂ (665 mg, 5.7 mmol), Zn powder (364 mg, 5.7 mmol), Pd₂(dba)₃ (520 mg, 0.57 mmol) and dppf (315 mg, 0.57 mmol). The mixture was heated in microwave at 130° C. for 35 min, then partitioned between EtOAc (200 mL) and water (50 mL). The org. phase was washed with water and brine, dried over anhydrous sodium sulfate and concentrated under vacuum. The residue was purified by column chromatography on silica gel (eluted with Petroleum Ether/EtOAc=10:1-1:1) to afford N-benzhydryl-6-cyano-4-methoxynicotinamide. LC/MS (m/z): 344 (M+H)⁺.

### Step 2: N-benzhydryl-4-methoxy-6-(1H-tetrazol-5-yl)nicotinamide

To a solution of N-benzhydryl-6-cyano-4-methoxynicotinamide (0.8 g, 2.3 mmol) in DMF (10 mL) were added NaN₃ (227 mg, 3.5 mmol) and NH₄Cl (189 mg, 3.5 mmol). The mixture was stirred at 110 C for 1.5 h. After cooling, the reaction mixture was partitioned between EtOAc (200 mL) and water (50 mL). The org. phase was washed with water (50 mL) and brine (50 mL), dried over anhydrous sodium sulfate and concentrated under vacuum to afford N-benzhydryl-4-methoxy-6-(1H-tetrazol-5-yl)nicotinamide. LC/MS (m/z): 387 (M+H)⁺.

### Step 3: 2-(5-(5-(benzhydrylcarbamoyl)-4-hydroxypyridin-2-yl)-1H-tetrazol-1-yl)acetic acid

To a solution of N-benzhydryl-4-methoxy-6-(1H-tetrazol-5-yl)nicotinamide (0.9 g, 2.3 mmol) in DMF (10 mL) was added K₂CO₃ (965 mg, 7 mmol) and tert-butyl 2-bromoacetate (0.9 g, 4.6 mmol). The mixture was stirred at rt for 16 h, then partitioned between EtOAc (200 mL) and water (50 mL). The org. phase was washed with water and brine (50 mL), dried over anhydrous sodium sulfate and concentrated under vacuum. The residue was purified by column chromatography on silica gel (eluted with Petroleum ether/EtOAc=5:1-3:1) to afford tert-butyl 2-(5-(5-(benzhydrylcarbamoyl)-4-methoxypyridin-2-yl)-1H-tetrazol-1-yl)acetate.

¹H NMR (CDCl₃, 400 MHz) δ 9.26 (s, 1H), 8.26 (brd, J=7.6 Hz, 1H), 8.07 (s, 1H), 7.37-7.31 (m, 10H), 6.46 (d, J=7.6 Hz, 1H), 5.64 (s, 2H), 4.13 (s, 3H), 1.40 (s, 9H). LC/MS (m/z): 501 (M+H)⁺.

To a flask (50 mL) were added tert-butyl 2-(5-(5-(benzhydrylcarbamoyl)-4-methoxypyridin-2-yl)-1H-tetrazol-1-yl)acetate (60 mg, 0.12 mmol) and HBr-AcOH (40%, 8 mL), and the mixture was stirred at 80° C. for 40 min, then concentrated under vacuum. The residue was purified by prep. HPLC to afford 2-(5-(5-(benzhydrylcarbamoyl)-4-hydroxypyridin-2-yl)-1H-tetrazol-1-yl)acetic acid. ¹H NMR (CD₃OD, 400 MHz) δ 8.89 (s, 1H), 7.49 (s, 1H), 7.36-7.28 (m, 10H), 6.40 (s, 1H), 5.71 (s, 2H). LC/MS (m/z): 431 (M+H)⁺. Human HIF-PHD2 IC₅₀: 18.39 nM.

### EXAMPLE 5

### ethyl 1-((5-((bis(4-chlorophenyl)methyl)carbamoyl)-4-hydroxypicolinamido)methyl) cyclopropanecarboxylate

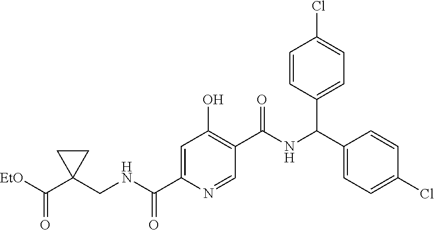 [SMILES: CCOC(=O)C1(CNC(=O)c2cc(O)c(C(=O)NC(c3ccc(Cl)cc3)c3ccc(Cl)cc3)cn2)CC1]

### Step 1: tert-butyl 6-chloro-4-methoxynicotinate

A suspension of 6-chloro-4-methoxynicotinic acid (12 g, 64 mmol) in SOCl₂ (60 ml) was refluxed for 4 hours. The resulting solution was concentrated under vacuum, and the residue was azeotroped with toluene (30 ml) to afford a yellow solid, which was added into tBuOH (50 ml) and stirred at room temperature overnight. The reaction mixture partitioned between aq.NaOH (200 ml, 5%) and DCM (100 mL), and the aq. phase was extracted with DCM (2×100 mL). The organic layers were washed with brine (100 mL), dried over Na₂SO₄ and concentrated to afford tert-butyl 6-chloro-4-methoxynicotinate as yellow solid. ¹H NMR (CDCl₃, 400 MHz) δ 8.60 (s, 1H), 6.87 (s, 1H), 3.92 (s, 3H), 1.54 (s, 9H). LC/MS (m/z): 244 (M+H)⁺.

### Step 2: 5-tert-butyl 2-methyl 4-methoxypyridine-2,5-dicarboxylate

To an autoclave were added a solution of tert-butyl 6-chloro-4-methoxynicotinate (5.6 g, 29 mmol) in MeOH (500 ml), PdCl₂(dppf) (2.2 g, 2.9 mmol) and NaOAc (4.8 g, 58 mmol). The air in the autoclave was replaced with carbon monoxide, and the pressure was adjusted to 30 atm. The reaction mixture was stirred at 120° C. for 16 hours. After cooling, the mixture was filtered, and the filtrate was concentrated under vacuum. The residue was purified by column chromatography on silica gel (eluted with petroleum ether/EtOAc=3:1) to afford 5-tert-butyl 2-methyl 4-methoxypyridine-2,5-dicarboxylate as yellow solid. ¹H NMR (DMSO-d₆, 400 MHz) δ 8.68 (s, 1H), 7.68 (s, 1H), 3.95 (s, 3H), 3.87 (s, 3H), 1.50 (s, 9H). LC/MS (m/z): 268 (M+H)⁺.

### Step 3: 5-(tert-butoxycarbonyl)-4-methoxypicolinic acid

To a solution of 5-tert-butyl 2-methyl 4-methoxypyridine-2,5-dicarboxylate (2.5 g, 9.4 mmol) in MeOH (30 mL) was added aq. NaOH (3 M, 6 mL, 18 mmol). The resulting mixture was stirred at room temperature for 4 hours. When TLC showed the reaction completed, the reaction mixture was concentrated under vacuum, and the residue was diluted with water (5 mL) and acidified with diluted hydrochloric acid to pH=3-4. The precipitate was collected by suction to give 5-(tert-butoxycarbonyl)-4-methoxypicolinic acid as white solid. ¹H NMR (DMSO-d₆, 400 MHz) δ 8.68 (s, 1H), 7.69 (s, 1H), 3.95 (s, 3H), 1.51 (s, 9H). LC/MS (m/z): 254 (M+H)⁺.

### Step 4: tert-butyl 6-(((1-(ethoxycarbonyl)cyclopropyl)methyl)carbamoyl)-4-methoxynicotinate

To a solution of 5-(tert-butoxycarbonyl)-4-methoxypicolinic acid (900 mg, 3.6 mmol) in DCM (20 mL) were added ethyl 1-(aminomethyl)cyclopropanecarboxylate (770 mg, 5.4 mmol), TBTU (2.1 g, 6.4 mmol) and TEA (1.4 g, 10.7 mmol). The mixture was stirred at room temperature for 5 hours. When TLC showed the reaction completed, the mixture was diluted with water (80 ml), extracted with DCM (3×80 mL). The organic layers were washed with brine, dried over Na₂SO₄ and concentrated. The crude product was purified by prep. TLC (EtOAc/PE=1:1) to affordethyl 3-(5-(benzhydrylcarbamoyl)-4-methoxypicolinamido)propanoate as white solid. ¹H NMR (CDCl₃, 400 MHz) δ 8.74 (s, 1H), 8.66 (m, 1H), 7.75 (s, 1H), 4.15 (q, J=6.9 Hz, 2H), 3.98 (s, 3H), 3.58 (d, J=6.2 Hz, 2H), 1.55 (s, 9H), 1.28-1.21 (m, 5H), 1.01-0.95 (m, 2H). LC/MS (m/z): 379 (M+H)⁺.

### Step 5:: 6-(((1-(ethoxycarbonyl)cyclopropyl)methyl)carbamoyl)-4-methoxynicotinic acid

To a solution of tert-butyl 6-(((1-(ethoxycarbonyl)cyclopropyl)methyl)carbamoyl)-4-methoxynicotinate (980 mg, 2.6 mmol) in DCM (30 mL) was added TFA (11.8 g, 104 mmol). The reaction mixture was stirred at room temperature for 4 hours. When TLC showed the reaction completed, the reaction mixture was concentrated and azeotroped with DCE (30 ml) to afford 6-(((1-(ethoxycarbonyl)cyclopropyl)methyl)carbamoyl)-4-methoxynicotinic acid as a yellow oil. ¹H NMR (DMSO-d₆, 400 MHz) δ 8.76-8.70 (m, 2H), 7.74 (s, 1H), 4.08 (q, J=7.0 Hz, 2H), 4.00 (s, 3H), 3.58 (d, J=6.0 Hz, 2H), 1.18 (t, J=7.0 Hz, 3H), 1.10 (m, 2H), 1.01 (m, 2H). LC/MS (m/z): 323 (M+H)⁺.

### Step 6: ethyl 1-((5-((bis(4-chlorophenyl)methyl)carbamoyl)-4-methoxypicolinamido)methyl) cyclopropanecarboxylate

To a solution of 6-(((1-(ethoxycarbonyl)cyclopropyl)methyl)carbamoyl)-4-methoxynicotinic acid (50 mg, 0.16 mmol) in DCM (4 mL) were added bis(4-chlorophenyl)methylamine (80 mg, 0.32 mmol), TBTU (62 mg, 0.19 mmol) and DIPEA (62 mg, 0.48 mmol). The mixture was stirred at room temperature overnight. When TLC showed the reaction completed, the mixture was diluted with water (15 ml), extracted with DCM (3×20 mL). The organic layer was washed with brine, dried over Na₂SO₄ and concentrated. The crude product was purified by prep. TLC (DCM/MeOH=15:1) to afford ethyl 1-((5-((bis(4-chlorophenyl)methyl)carbamoyl)-4-methoxypicolinamido)methyl) cyclopropanecarboxylate as a white solid. ¹H NMR (400 MHz, CDCl₃) δ 9.17 (s, 1H), 8.72 (t, J=6.2 Hz, 1H), 8.14 (d, J=7.5 Hz, 1H), 7.83 (s, 1H), 7.32 (d, J=8.4 Hz, 4H), 7.21 (d, J=8.4 Hz, 4H), 6.38 (d, J=7.5 Hz, 1H), 4.18 (q, J=7.1 Hz, 2H), 4.06 (s, 3H), 3.62 (d, J=6.4 Hz, 2H), 1.31-1.26 (m, 5H), 1.04-0.99 (m, 2H). LC/MS (m/z): 556 (M+H)⁺.

### Step 7: ethyl 1-((5-((bis(4-chlorophenyl)methyl)carbamoyl)-4-hydroxypicolinamido)methyl) cyclopropanecarboxylate

To a solution of ethyl 1-((5-((bis(4-chlorophenyl)methyl)carbamoyl)-4-methoxypicolinamido)methyl) cyclopropanecarboxylate (60 mg, 0.11 mmol) and NaI (83 mg, 0.55 mmol) in CH₃CN (6 mL) was added TMSC₁ (120 mg, 1.1 mmol). The reaction mixture was stirred at 45° C. for 4 hours. When TLC showed the reaction completed, the reaction mixture was diluted with EtOH (15 mL), the solution was concentrated under vacuum to remove solvents. The residue was re-dissolved in EtOAc (40 mL), washed with sat. aqueous Na₂SO₃ and brine, dried over Na₂SO₄ and concentrated to afford ethyl 1-((5-((bis(4-chlorophenyl)methyl)carbamoyl)-4-hydroxypicolinamido)methyl)cyclopropanecarboxylate as a white solid. ¹H NMR (DMSO-d₆, 400 MHz) δ 8.92 (br s, 1H), 8.37 (s, 1H), 7.39 (d, J=8.2 Hz, 4H), 7.30 (d, J=8.2 Hz, 4H), 7.17 (br s, 1H), 6.27 (d, J=7.9 Hz, 1H), 4.04 (q, J=6.9 Hz, 2H), 3.50 (d, J=5.1 Hz, 2H), 1.13 (t, J=6.9 Hz, 3H), 1.08 (s, 2H), 0.97 (s, 2H). LC/MS (m/z): 542 (M+H)⁺. Human HIF-PHD2 IC₅₀: 183.8 nM.

### Biological Assays

The exemplified compounds of the present invention have been found to inhibit the hydroxylation of a HIF peptide by PHD2 and exhibit IC₅₀ values ranging between 0.1 nanomolar to 10 micromolar. Select examples of assays that may be used to detect favorable activity are disclosed in the following publications: Oehme, F., et al., Anal. Biochem. 330:74-80 (2004); Hirsilä, M, et al., J. Bio. Chem. 278 (33): 30772-30780 (2005); Hyunju, C., et al., Biochem. Biophys. Res. Comm. 330 275-280 (2005); and Hewitson, K. S., et al., Methods in Enzymology, (Oxygen Biology and Hypoxia); Elsevier Publisher (2007), pg. 25-42 (ISSN: 0076-6879).

The biological activity of the present compounds may be evaluated using assays described herein below:

To each well of a 384-well plate, 1 μL of test compounds in DMSO (final concentration ranging from 0.3 nM to 10 μM) were added into 20 μl of assay buffer (50 mM Tris pH 7.4/0.01% Tween-20/0.1 mg/ml bovine serum albumin/10 μM ferrous sulfate/1 mM sodium ascorbate/20 μg/ml catalase) containing 0.15 μg/ml FLAG-tagged full length PHD2 expressed in and purified from baculovirus-infected Sf9 cells. After a 5 min preincubation at room temperature, the enzymatic reactions were initiated by the addition of 4 μL of substrates final concentrations of 0.2 μM 2-oxoglutarate and 0.5 μM HIF-la peptide biotinyl-DLDLEMLAPYIPMDDDFQL (SEQ ID NO:1)1. After incubation for 45 minutes at room temperature, the reactions were terminated by the addition of a 25 μL quench/detection mix to a final concentration of 1 mM ortho-phenanthroline, 0.1 mM EDTA, 0.5 nM anti-(His)6 LANCE reagent (Perkin-Elmer Life Sciences), 100 nM AF647-labeled streptavidin (Invitrogen), and 2 μg/ml (His)6-VHL complex {S. Tan Protein Expr. Purif. 21, 224-234 (2001)} and the signals were developed for 30 minutes at room temperature. The ratio of time resolved fluorescence signals at 665 and 620 nm was determined, and percent inhibition was calculated relative to the high control samples (DMSO treated) run in parallel, after background subtraction. Inhibition of the catalytic activity of HIF-PHD1 and HIF-PHD3 can be determined similarly, except for HIF-PHD3, final concentrations of 4 μM 2-oxoglutarate is used during the reaction.

What is claimed is:

1. A compound of formula I or a stereoisomer thereof, or a pharmaceutically acceptable salt thereof: 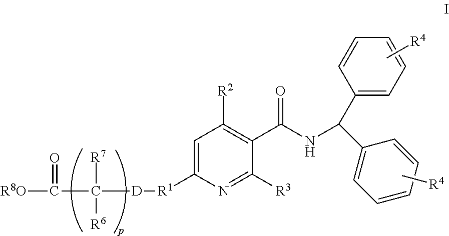
R¹ is —CONR⁵—, C₃₋₁₂cycloalkyldiyl, or a heteroaryldiyl selected from isoxazolyldiyl, tetrazolyldiyl, pyrazolyldiyl, imidazolyldiyl, oxazolyldiyl, thiazolyldiyl, pyridinyldiyl, pyradizinyldiyl, and pyrimidinyldiyl;
R⁵ is hydrogen, C₁₋₃alkyl, or C₁₋₃alkoxy;
R⁸ is hydrogen or C₁₋₃alkyl;
p is 0, 1, 2 or 3;
R² and R³ are each independently selected from hydrogen, hydroxy, —OR, —OCOR, —OCOOR, —OCONHR, and C₁₋₆alkyl;
R is independently selected from hydrogen, C₁₋₁₀ alkyl, —C₁₋₅ alkylaryl, —CR′R′—OCO-C₁-10 alkyl, and —CR′R′—OC₀₋₁₀—C₁₋₁₀ alkyl;
R′ and R″ are independently selected from hydrogen and C₁₋₁₀ alkyl;
D is selected from a bond, C₃₋₁₂cycloalkyldiyl, C₃₋₁₂cycloheteroalkyldiyl, aryldiyl, and heteroaryldiyl;
R⁴, R⁶, and R⁷ are each independently selected from
hydrogen,
halogen,
C₁-10 alkyl,
C₂-10 alkenyl,
C₂-10 alkynyl,
C₁₋₁₀ alkylamino,
arylC₀₋₁₀ alkyl,
C₃₋₈ cycloalkyl C₀₋₁₀ alkyl,
C₃₋₈ heteroaryl C₀-10 alkyl,
C₃₋₈ heterocycloalkyl C₀₋₁₀ alkyl,
C₁₋₁₀ alkoxy, and
hydroxyC₀-10alkyl, wherein R⁶ and R⁷ may optionally join together with the carbon to which they are attached to form a 3 to 8 membered ring;
wherein R⁴, R⁶, R⁷, and D are each optionally substituted with 0, 1, or 2 R⁹ substituents selected from:
hydrogen,
halogen,
(carbonyl)₀₋₁C₁₋₁₀ alkyl,
(carbonyl)₀₋₁C₂₋₁₀ alkenyl,
(carbonyl)₀₋₁C₂₋₁₀ alkynyl,
amino C₀₋₁₀ alkyl,
C₁₋₁₀ alkylamino C₀₋₁₀ alkyl,
cyano,
nitro,
C₁₋₆haloalkyl,
perfluoroC₁₋₆alkyl, and
perfluoroC₁₋₆alkoxy.

2. A compound according to claim 1 of formula II or stereoisomers thereof, or pharmaceutically acceptable salts thereof: 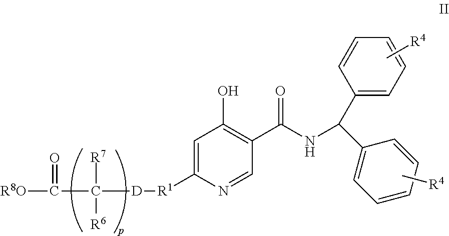
R¹ is —CONR⁵— or a heteroaryldiyl selected from isoxazolyldiyl, tetrazolyldiyl, pyrazolyldiyl, imidazolyldiyl, oxazolyldiyl, thiazolyldiyl, pyridinyldiyl, pyradizinyldiyl, and pyrimidinyldiyl;
R⁵ is hydrogen, C₁₋₃alkyl, or C₁₋₃alkoxy;
R⁸ is hydrogen or C₁₋₃alkyl;
p is 0, 1, 2 or 3;
D is selected from a bond and C₃₋₁₂cycloalkyldiyl, and
R⁴, R⁶, and R⁷ are each independently selected from
hydrogen,
halogen,
C₁₋₁₀ alkyl, and
hydroxyC₀-10alkyl, wherein R⁶ and R⁷ may optionally join together with the carbon to which they are attached to form a 3 to 8 membered ring.

3. A compound according to claim 2 of formula II or stereoisomers thereof, or pharmaceutically acceptable salts thereof, wherein:
R¹ is —CONH— or a heteroaryldiyl selected from tetrazolyldiyl, and pyrazolyldiyl;
R⁵ is hydrogen;
R⁸ is hydrogen or C₁₋₃alkyl;
p is 0, 1, 2 or 3;
D is selected from a bond, cyclopropyl, cyclobutyl, and bicylco[1.1.1]pentyl; and
R⁴, R⁶, and R⁷ are each independently selected from
hydrogen,
halogen,
C₁₋₃ alkyl, and
hydroxy, wherein R⁶ and R⁷ may optionally join together with the carbon to which they are attached to form a 3 to 8 membered ring.

4. A compound according to claim 4 of formula II or stereoisomers thereof, or pharmaceutically acceptable salts thereof, wherein:
R⁸ is hydrogen, methyl, or ethyl; R⁴ is hydrogen or chloro; R⁶ and R⁷ are each independently selected from hydrogen, methyl, and hydroxy, wherein R⁶ and R⁷ may optionally join together with the carbon to which they are attached to form a 3 membered ring.

5. A compound which is:
1-(5-(Benzhydrylcarbamoyl)-4-hydroxypyridin-2-yl)-1H-pyrazole-4-carboxylic acid;
methyl 3-(5-(benzhydrylcarbamoyl)-4-methoxypicolinamido)propanoate;
methyl 3-(5-(benzhydrylcarbamoyl)-4-hydroxypicolinamido)-2-methylpropanoate;
ethyl 1-((5-(benzhydrylcarbamoyl)-4-hydroxypicolinamido)methyl)cyclopropanecarboxylate;
ethyl 3-(5-(benzhydrylcarbamoyl)-4-hydroxypicolinamido)-2,2-dimethylpropanoate;
(1S,2S)-ethyl 2-(5-(benzhydrylcarbamoyl)-4-hydroxypicolinamido)cyclopropanecarboxylate;
(1R,2R)-ethyl 2-(5-(benzhydrylcarbamoyl)-4-hydroxypicolinamido)cyclopropanecarboxylate;
trans-3-(5-(benzhydrylcarbamoyl)-4-hydroxypicolinamido)cyclobutanecarboxylic acid;
3-(5-(benzhydrylcarbamoyl)-4-hydroxypicolinamido)propanoic acid;
3-(5-(benzhydrylcarbamoyl)-4-hydroxypicolinamido)-2-methylpropanoic acid;
1-((5-(benzhydrylcarbamoyl)-4-hydroxypicolinamido)methyl)cyclopropanecarboxylic acid;
4-(5-(benzhydrylcarbamoyl)-4-hydroxypicolinamido)butanoic acid;
3-(5-(benzhydrylcarbamoyl)-4-hydroxypicolinamido)-2-hydroxypropanoic acid;
3-(5-(benzhydrylcarbamoyl)-4-hydroxypicolinamido)-2,2-dimethylpropanoic acid;
(1S,2S)-2-(5-(benzhydrylcarbamoyl)-4-hydroxypicolinamido)cyclopropanecarboxylic acid;
(1R,2R)-2-(5-(benzhydrylcarbamoyl)-4-hydroxypicolinamido)cyclopropanecarboxylic acid;
3-(5-(benzhydrylcarbamoyl)-4-hydroxypicolinamido)bicyclo[1.1.1]pentane-1-carboxylic acid;
2-(5-(5-(benzhydrylcarbamoyl)-4-hydroxypyridin-2-yl)-1H-tetrazol-1-yl)acetic acid; and
ethyl 1-((5-((bis(4-chlorophenyl)methyl)carbamoyl)-4-hydroxypicolinamido)methyl) cyclopropanecarboxylate; or
a stereoisomer thereof or a pharmaceutically acceptable salt thereof.

6. A compound of claim 1 or a pharmaceutically acceptable salt thereof, for use as a medicament.

7. A compound of claim 1 or a pharmaceutically acceptable salt thereof, for the treatment of conditions mediated by HIF prolyl hydroxylase.

8. A pharmaceutical composition comprising a compound of claim 1 and pharmaceutically acceptable carrier.

9. A method of enhancing endogenous production of erythropoietin in a mammal which comprises administering to the mammal an amount of a compound of claim 1, or a pharmaceutically acceptable salt or solvate thereof, that is effective for enhancing endogenous production of erythropoietin.

10. A method for the prevention or treatment of anemia in a mammal which comprises administering to the mammal an effective amount of a compound of claim 1, or a pharmaceutically acceptable salt or solvate thereof.

11. Use of a compound of claim 1, or a pharmaceutically acceptable salt or solvate thereof, in the manufacture of medicaments for the treatment of conditions mediated by HIF prolyl hydroxylase.
